(19) United States
(12) Patent Application Publication
(10) Pub. No.: US 2002/0029048 A1
(43) Pub. Date: Mar. 7, 2002

(54) Endovascular fastener and grafting apparatus and method

(76) Inventor: Arnold Miller, chestnut Hill, MA (US)

Correspondence Address: Pandiscio & Pandiscio, 470 Totten Pond Road, Waltham, MA, 02451-1914, US

(21) Appl. No.: 09/944,899

(22) Filed: Aug. 31, 2001

Related U.S. Application Data
(60) Provisional application No. 60/229,788, filed on Sep. 1, 2000.

(51) Int. Cl. A61B 17/08

(52) U.S. Cl. 606/138; 606/213

(57) ABSTRACT

A endovascular fastener and grafting apparatus preferably made from a shape memory alloy is provided which can access internal tissue or other synthetic material by catheter delivery through an endovascular pathway. After the fastener is deployed through layers of tissue or other material, it assumes a shape that automatically applies to the layers of tissue or other material an appropriate hemostatic compression which is relatively independent of tissue or material thickness. The fastener is a suitable replacement for conventional nonbio-absorbable sutures and staples in certain clinical applications. The shape, method of deployment and low force requirements make the disclosed apparatus suitable for standard endovascular surgical procedures where access to the deployment site is limited. A method for deploying the endovascular fastener and grafting apparatus is also provided.

### BACKGROUND OF THE INVENTION

Historically, living tissue has been most commonly surgically repaired by thread, such as a suture, introduced by a pointed metal needle and tied with just enough tension to establish hemostasis or control of bleeding by compressing the tissue. Correct tension is established by the surgeon based on observation and judgment derived from extensive training. Excess tension can cause necrosis (the localized death of living tissue) and eventual failure of the repair.

An alternate method of joining tissue using metal staples has evolved over the last 90 years to a point where specialized staples for both skin and internal tissue closure are in common use today. The staples, which have sharp points for penetrating tissue, are formed in place by delivery instruments which bend them to a permanent shape suitable for tissue retention. The delivery instruments include mechanisms, such as an anvil, which control to some extent the relationship between tissue and staple, including the compression necessary to control bleeding. To the extent that they do so, surgeon skill is less of a factor in successful wound closure.

For conventional surgery, the clinical results for suturing and stapling are essentially the same, but both have their disadvantages. Sutures are suitable for all types of wound closure, but require that the surgeon have adequate access to the wound site and possess the skill to choose and apply the suture correctly. Conventional staples can also be appropriate for internal use, but require that a strong, rigid anvil be placed behind the tissues to be joined. Furthermore, the application of staples requires that there be enough space for an instrument, which can produce the necessary force to form the staple against the anvil. Stapling, however, is generally faster and, as previously noted, requires a lower level of skill.

The recent development of a beneficial, less invasive technique for gall bladder removal has suggested the feasibility of other abdominal procedures, such as bowel and hernia repair, that require the remote application of an internal fastener. As a result, less invasive instruments have been developed for both suturing and stapling remotely from the wound site by the surgeon. At the same time, patient benefit considerations are driving the development of less invasive techniques for a full range of abdominal and thoracic procedures including coronary artery bypass and valve replacement.

To date, stapling has proven to be more suitable for less invasive surgery than suturing. Instruments developed for that purpose approximately replicate the functions of stapler developed for open surgery and are approximately as easy to use. Instruments developed for less invasive suturing, on the other hand, are slow and cumbersome and do not solve the essential problem of tensioning the suture and tying the knot remotely. Sutures will find limited use in less invasive surgery but it is most likely that related wound closure problems beyond the capability of conventional staples will be solved by innovative mechanical fasteners which can more easily be remotely applied.

For instance, a new fastener has been designed for a less invasive hernia repair in which a synthetic mesh is used to reinforce the repair by anchoring it to surrounding tissue. Suturing is feasible but difficult. Conventional stapling is not feasible because an anvil cannot access the distal side of the tissue. The new fastener has the shape of a coil spring with the wire sharpened at one end and has been used successfully to attach the mesh by screwing the coil through it into the tissue. This new fastener can access the wound site through a small port in the abdominal wall. This fastener, however, does not produce compression upon the synthetic and natural tissue layers and thus does not produce hemostasis because the fastener is screwed into the wound site in its natural shape. Because this fastener does not create hemostasis, it may not be suitable for a wide range of surgical applications.

Other surgical fasteners have been fabricated from shape memory alloy. U.S. Pat. No. 4,485,816 to Krumme discloses a shape-memory surgical staple that uses an electric current to heat the staple to make it close. U.S. Pat. No. 5,002,562 to Pyka et al. discloses a fastener made from shape memory alloy that has the shape of a suturing loop in its undeformed shape. As noted above, however, sutures and staples are not always desirable for all surgical applications.

It is believed that other applications exist or will be identified for fastening layers of tissue where anvil access is not practical and where compression must be applied to the tissue to achieve hemostasis. For example, these criteria apply to the attachment of a graft more or less at right angles to another, larger, blood vessel (“end to side” anastomosis) such as the aorta for vascular bypass purposes. The availability of a less invasive vascular bypass procedure implies a significant patient benefit. Another example is the use of the fastener in endovascular procedures to attach a graft within large vessels such as the aorta, iliac or femoral arteries to repair aneurysms and occlusions. Stents, which are currently used for this purpose, are often insufficiently compliant to prevent leakage and consequent failure of the repair. Direct fixation of the graft to the inner wall of the vessel by the fasteners described herein may overcome this inherent problem of current techniques for endovascular repair.

What is desired, therefore, is a mechanical fastener and deployment instrument that can access internal tissue through a small surgical access port or incision and that can be applied conveniently and remotely.

### SUMMARY OF THE INVENTION

Accordingly, an object of the present invention is to provide a surgical fastener that can access internal tissue through a small surgical access port or incision.

It is a further object of the present invention to provide a surgical fastener that can be applied remotely.

It is yet another object of the present invention to provide a surgical fastener that uses the superelastic properties of a shape memory alloy without having to apply heat to the fastener.

It is still another object of the present invention to provide a deployment instrument that can be used to deploy the surgical fasteners of above.

These objects of the invention are achieved by a surgical fastener preferably made from a shape memory alloy that accesses internal tissue or other synthetic material through a small surgical access port or incision. After the fastener is deployed through layers of tissue, it assumes a shape that automatically applies to the layers of tissue an appropriate hemostatic compression which is relatively independent of tissue thickness. The fastener is a suitable replacement for conventional non bio-absorbable sutures and staples in certain clinical applications. Its shape, method of deployment and low force requirements make it suitable for standard surgical procedures and especially suitable for laparoscopic and other less invasive surgery where access to the wound site is limited including endovascular surgery. The invention is expected to be especially useful for attaching synthetic grafts to an aorta.

In one form of the invention, there is provided apparatus for inserting a surgical fastener through a plurality of portions of material from within an endovascular pathway, the apparatus comprising:

a surgical fastener having first and second ends and made from a material which enables the fastener to be transformed from a first stressed elongate shape to a second unstressed shape upon the release of the fastener from a stressed condition, the first stressed elongate shape of the fastener enabling the first end to be extended through a plurality of layers of material, and with the second shape of the fastener being in the form of a spring with a plurality of coils around a spring axis, with the coils being spring biased towards each other along the spring axis with sufficient axial force so as to enable coils on opposite sides of layers to clamp the layers of material together along the spring axis;

a delivery tube having third and fourth ends, first and second tube portions adjacent to the third and fourth ends, respectively, and forming a longitudinal axis between the third and fourth ends, the delivery tube including a material which enables transformation from a third stressed elongate shape to a fourth unstressed shape upon the release from a stressed condition to an unstressed condition, the third stressed elongate shape enabling the third end to be extended through an endovascular pathway, with the fourth unstressed shape being formed with the first and second tube portions being configured at an angle to one another;

delivery tube deployment means being configurable between a first position and a second position, the first position of the delivery tube deployment means restraining the delivery tube in the third stressed elongate shape, and the second position of the delivery tube deployment means releasing the delivery tube in the fourth unstressed shape;

penetration means adjacent the third end of the delivery tube, the penetration means being configured to pierce through a vascular structure in the endovascular pathway; and

insertion means adjacent to the first end of the delivery tube, the insertion means being configured to place the surgical fastener through the vascular structure pierced by the penetration means.

In another form of the invention, there is provided a method for inserting a surgical fastener through a plurality of portions of material from within an endovascular pathway, the method comprising:

providing apparatus for inserting a surgical fastener through a plurality of portions of material from within an endovascular pathway, the apparatus comprising:

a surgical fastener having first and second ends and made from a material which enables the fastener to be transformed from a first stressed elongate shape to a second unstressed shape upon the release of the fastener from a stressed condition, the first stressed elongate shape of the fastener enabling the first end to be extended through a plurality of layers of material, and with the second shape of the fastener being in the form of a spring with a plurality of coils around a spring axis, with the coils being spring biased towards each other along the spring axis with sufficient axial force so as to enable coils on opposite sides of layers to clamp the layers of material together along the spring axis;

a delivery tube having third and fourth ends, first and second tube portions adjacent to the third and forth ends, respectively, and forming a longitudinal axis between the third and fourth ends, the delivery tube including a material which enables transformation from a third stressed elongate shape to a fourth unstressed shape upon the release from a stressed condition to an unstressed condition, the third stressed elongate shape enabling the third end to be extended through an endovascular pathway, with the fourth unstressed shape being formed with the first and second tube portions being configured at an angle to one another;

delivery tube deployment means being configurable between a first position and a second position, the first position of the delivery tube deployment means restraining the delivery tube in the third stressed elongate shape, and the second position of the delivery tube deployment means releasing the delivery tube in the fourth unstressed shape;

penetration means adjacent the third end of the delivery tube, the penetration means being configured to pierce through a vascular structure in the endovascular pathway; and

insertion means adjacent to the first end of the delivery tube, the insertion means being configured to place the surgical fastener through the vascular structure pierced by the penetration means;

placing the delivery tube adjacent the vascular structure, with the delivery tube being configured in the third stressed elongate shape;

deploying the delivery tube from the third elongate shape to said fourth elongate shape with the delivery tube deployment means, the deployment of the delivery tube placing the third end adjacent to the vascular structure in the endovascular pathway;

penetrating the vascular structure in the endovascular pathway with the penetration means, the penetration of the vascular structure being performed at the third end of the delivery tube; and

inserting the surgical fastener through the plurality of portions of material using the insertion means, the insertion of the surgical fastener being performed from inside of the vascular structure.

The above and other features of the invention, including various novel details of construction and combinations of parts and method steps, will now be more particularly described with reference to the accompanying drawings and pointed out in the claims. It will be understood that the particular devices and method steps embodying the invention are shown by way of illustration only and not as limitations of the invention. The principles and features of this invention may be employed in various and numerous embodiments without departing from the scope of the invention.

### BRIEF DESCRIPTION OF THE DRAWINGS

These and other objects and features of the present invention will be more fully disclosed or rendered obvious by the following detailed description of the preferred embodiments of the invention, which are to be considered together with the accompanying drawings wherein like numbers refer to like parts, and further wherein:

FIGS. 1A, 1B and 1C are an isometric view and two side views, respectively, of the first embodiment of the surgical fastener in accordance with the invention;

FIG. 2 is an isometric view of the second embodiment of the surgical fastener in accordance with the invention;

FIG. 3 is a side cutaway view of the second embodiment of the surgical fastener of FIG. 2 in accordance with the invention;

FIG. 4 a side cutaway view of the third embodiment of the surgical fastener in accordance with the invention;

FIGS. 5A-5F are front cutaway views of a deployment instrument showing the insertion of the surgical fastener of FIG. 1;

FIGS. 6A-6F are front isometric views of another embodiment of a deployment instrument showing the insertion of a surgical fastener;

FIG. 7 is a front isometric view of the deployment instrument of FIGS. 5A-5F as it is shipped;

FIG. 8 is a front cutaway view of the deployment instruments of FIGS. 5A-5F and 6A-6F;

FIGS. 9A-9D are side cutaway views showing the use of a deployment instrument with the surgical fastener of FIG. 2;

FIG. 10 is a diagrammatic view of apparatus for inserting a surgical fastener through a plurality of portions of material from within an endovascular pathway;

FIG. 11 is a diagrammatic view of the apparatus shown in FIG. 10 with a graft and a stent expanded in an aorta;

FIG. 12 is a diagrammatic view of the apparatus shown in FIGS. 10 and 11 with delivery tubes returning to an unstressed, preformed condition for penetration through the graft, stent and the aorta;

FIG. 13 is a diagrammatic view of the apparatus shown in FIGS. 10-12 with the delivery tubes in an unstressed, preformed configuration and positioned such that their ends penetrate through the wall of the aorta;

FIG. 14 is a diagrammatic view of the apparatus shown in FIGS. 10-13, with first ends of the surgical fasteners emerging from the ends of delivery tubes penetrating through the wall of the aorta;

FIG. 15 is a diagrammatic view of the apparatus shown in FIGS. 10-14, with delivery tubes withdrawn from second ends of the surgical fasteners such that the first and second ends of the surgical fasteners are biased closed toward one another;

FIG. 16 is a diagrammatic view of the distal end of the apparatus shown in FIG. 10 showing a closed configuration on a guide wire;

FIG. 17 is a diagrammatic view of the apparatus shown in FIG. 16 showing the outer endovascular graft delivery sheath partially withdrawn from the stent, with the stent surrounding the endovascular graft, which is partially withdrawn from the inner sheath, which is itself partially withdrawn from the delivery tubes;

FIG. 18 is a diagrammatic view of the apparatus shown in FIG. 17 with the graft and the stent extended and expanded over the ends of the delivery tubes (also shown in FIG. 11);

FIG. 19 is a diagrammatic view of the apparatus shown in FIG. 18, with delivery tubes returning to an unstressed, preformed configuration for penetration through the graft, stent and the aorta (also shown in FIG. 12);

FIG. 20 is a diagrammatic view of the apparatus shown in FIG. 19, with the delivery tubes in an unstressed, preformed configuration such that their ends penetrate through the wall of the aorta; and

FIG. 21 is another diagrammatic view of the apparatus shown in FIG. 20 with the delivery tubes penetrating through the wall of the aorta.

### DETAILED DESCRIPTION OF THE PREFERRED EMBODIMENTS

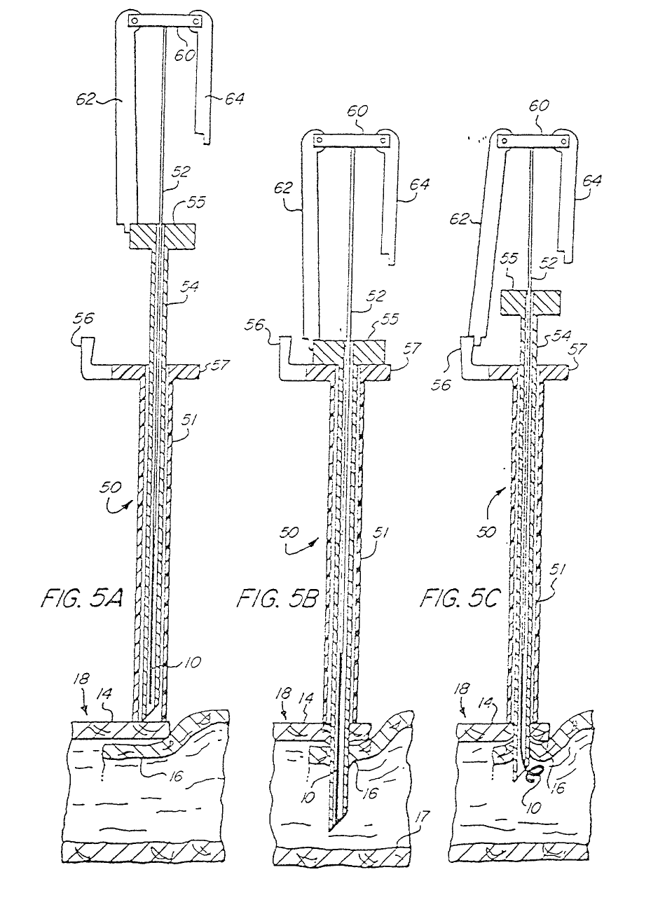

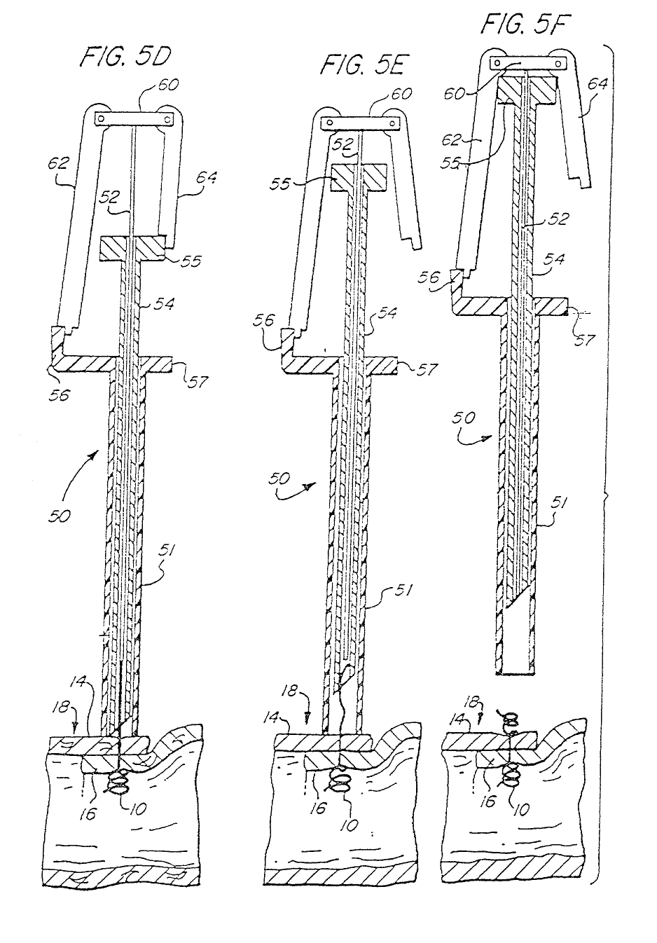

Surgical fasteners, each in accordance with the invention, are shown in FIGS. 1A-4. The surgical fastener is preferably a one piece metal or plastic element appropriately configured during manufacture to hold layers of tissue in compression. To apply the fastener, as shown in FIGS. 5A-5F, 6A-6F, and 9A-9D, a straight tube or needle included in a delivery mechanism is preferably used to hold and deflect the fastener from its final shape into a straight configuration. In application, the tube is either inserted through the tissue or held against the tissue to be joined and the fastener is pushed from the tube until the fastener penetrates the tissue and gradually assumes its original shape, trapping and compressing the layers of tissue 18 between its various elements.

In order to straighten the various surgical wire fasteners described herein without permanent deformation, a superelastic alloy of nickel and titanium is preferably used to make the fasteners. The fastener is preferably made from a commercial material Nitinol, which is referred to as a “shape memory alloy.” Superelasticity can be conveniently likened to memory. Although forced into a straight line after forming, the superelastic fastener is able to “remember” its former shape and to return to it when no longer constrained within a straight tube. Nitinol in superelastic form has an extremely high elastic limit, which allows large amounts of bending without permanent deformation. In general, Nitinol is capable of strain ratios of up to 8% without experiencing permanent deformation. For round wire, the fastener is designed to function within the limits of d/2R equal to or less than 0.08, where d is the diameter of the wire and R is the radius to which the wire is formed. It should be noted that the fastener described herein can be made from any material so long as it is adequately elastic. Preferably, the material has superelastic characteristics.

The preferred embodiment of the fastener 10, shown in FIGS. 1A-1C, is essentially that of the body of an extension spring having coils 12. At rest, the coils of this fastener 10 are spring biased towards each other so that a force is F_{A} required to effect separation of said coils. The force at which the coils just begin to separate is the preload value for the fastener. Additional force causes separation of the coils 12 as a function of the gradient of the fastener. Shown in FIG. 1C, layers of tissue 18 that are trapped between adjacent coils 12 of the fastener will be clamped with a force F₁ being substantially normal to the surface of the tissue 18 and having a value somewhat higher than the preload value of the fastener. This force, which is a function of fastener material, dimensions and winding technique, is chosen to insure hemostasis when vascular tissue is to be clamped. It should be noted that a compression spring could be used in place of an extension spring so long as the tissue is thick enough that it is compressed between the coils of the fastener once it is in place. The theory and practice of winding preloaded coils of metallic wire is routinely practiced in the manufacture of extension springs and is well known to those skilled in the art.

When the fastener of FIGS. 1A-1C is made of a superelastic material and the strain ratio limitation described above is observed, the fastener can be straightened to penetrate tissue 18 and then released to allow its coils to reform on both the proximate 14 and distal 16 sides of the tissue thereby clamping the tissue between two coils. The number of coils 12 is not especially critical. At least two full coils 12 are required and more, such as four coils, are preferable to make placement in the tissue less critical. The coils 12 preferably have a diameter of 3/16 to ¼ of an inch. Preferably, the end of the fastener inside of the body rests flush next to the adjacent coil so that the body will not be injured from the fastener end.

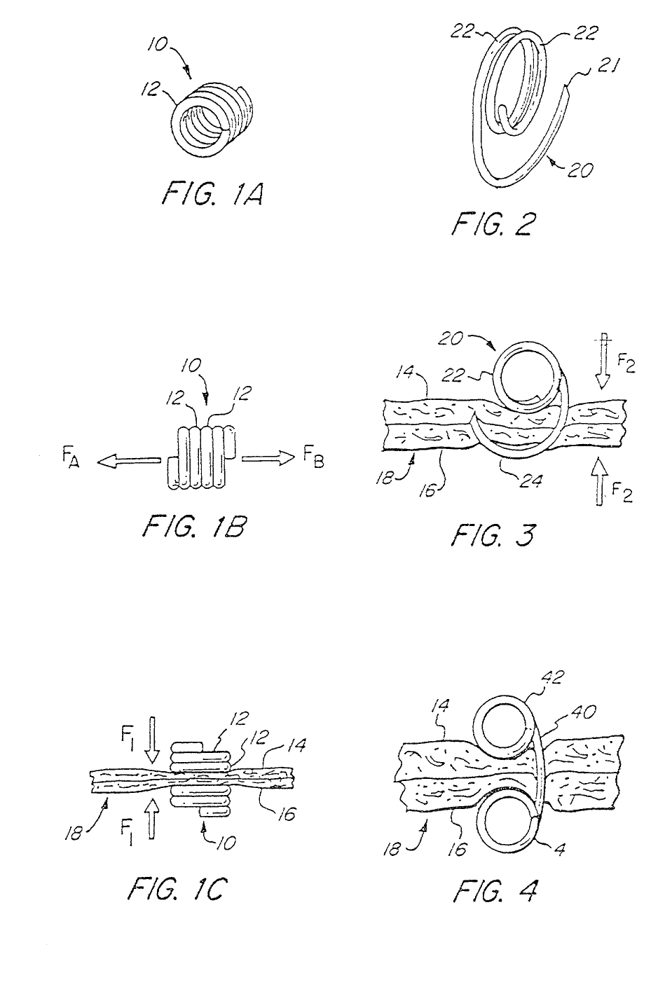

FIGS. 2 and 3 show another embodiment of the fastener 20 before and after installation in two layers 14, 16 of tissue 18. The presence of the tissue layers prevents the fastener from returning completely to its original state. The force required to spread the spring biased fastener apart by this amount therefore also represents the substantially normal compressive force F₂ applied to the layers of tissue 18. That force, which is a function of wire diameter and fastener geometry, is chosen by design to achieve homeostasis. Those parameters also determine the gradient or stiffness of the fastener as measured in terms of force F₂ versus deflection of the fastener. Since different tissue thicknesses produce different deflections, and therefore different compressive forces, the gradient must be sufficiently low to maintain reasonable hemostasis over the normal range of tissue thickness without inducing necrosis.

FIG. 2 is an isometric view of the fastener 20 shown schematically in FIG. 3. The lower coil 24 penetrates the tissue and curves in a half circle to re-enter the tissue layers. The upper coils 22 bear on the tissue and tend to trap it inside of the larger lower coil. The number of upper coils 22 can vary without altering the essential behavior of the fastener 20. Preferably, two or more coils 22 are used to help distribute clamping forces more uniformly about the lower coil thereby preventing misorientation of the fastener 20 in the tissue 18.

The fastener 40 in FIG. 4 has symmetrical coils to distribute stress uniformly on both sides of the tissues to be joined.

The fasteners in FIGS. 2-3 and 4 are similar to the fastener in FIGS. 1A-1C in that they are spring biased and use coils to apply pressure. The coils in FIGS. 2-3 and 4 each have an axis that is oriented substantially transverse to the direction that the fastener takes when it is in a straightened form, whereas the coils in FIGS. 1A-1C each have an have an axis that is substantially transverse to its straightened form.

The fasteners in FIGS. 1C, 3 and 4 all show a fastener clamping two layers of living tissue 18 which include a proximal layer 14 and a distal layer 16 of tissue. The fasteners described herein, however, can fasten any type of materials together, such as a graft or synthetic fibers which may be used as a substitute for tissue, or a combination thereof. The synthetic fibers, for example, may be a material such as Gore-Tex, Dacron or Teflon. Autogenous and nonautogenous human tissue, as well as animal tissue, may also be used.

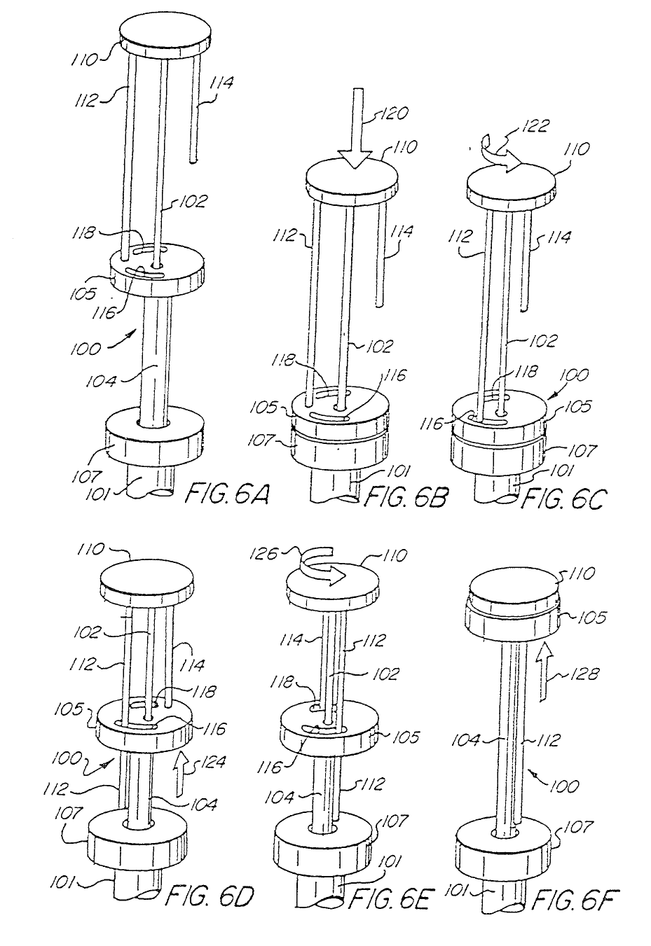

For all fasteners described above, the leading end 21 of the fastener, shown in FIG. 2, can be sharpened for ease of penetration either by cutting the wire on a bias or by tapering the end to a sharp point during manufacture of the fastener. The bias cut is commonly used to make sharp points on conventional staples and taper pointing is used to make a certain class of suture needles. Both techniques are well known to those skilled in the art. Other sharpening techniques such as trocar points may also be effectively applied to the fastener. Alternatively or additionally, a tube 154 of a delivery instrument 150 that houses the fastener, as shown in FIGS. 5A-5F and 6A-6F, can have a sharpened tip which is used to penetrate the tissue 18 prior to pushing the fastener from said tube.

A wide variety of fasteners can be designed within the scope of this invention for an equally wide variety of fastening purposes. Some of these shapes are shown in FIGS. 1A-4 and it should be apparent that other variations are both possible and likely as the invention becomes more widely applied.

The surgical fasteners described herein can also be used in applications that require the insertion of a fastener from the interior. For example, the fasteners can be used in endovascular procedures to attach a graft within large vessels such as the aorta or iliac arteries to repair aneurysms or occlusions.

FIGS. 5A-5F show a first embodiment of a deployment instrument 50 and the method for inserting the fastener. The deployment instrument 50 consists of a plunger 52 having a head portion 60, a needle 54 having a head portion 55, and a sleeve 51 having a head portion 57 and a stop 56. The plunger 52 fits slidingly inside a lumen of the needle 54, which fits slidingly inside of the sleeve 51. FIGS. 5A-5F show the fastener 10 being used to attach a graft 16 to a blood vessel having a first layer of tissue 14 and an opposite wall 17. The fasteners described herein, however, can be used for any layers of material or tissue. Furthermore, the delivery instrument 50 can deliver any of the fasteners described herein.

Depending on the situation, support for the lower membrane 16 will be required in order to insert the fastener 10. This normally will be the rigidity of the body tissue itself or a mechanical support which is provided separately, often as an integral part of the instrument that deploys the graft.

For the deployment instrument shown in FIGS. 5A-5D, the head portion 60 of the plunger 52 has two stops 62, 64 attached to it. One of the stops 62 pivotally engages of the head portion 55 of the needle 54 and also pivotally engages a stop 56 of the head portion 57 of the sleeve 51. The other stop 64 can engage the head portion 55 of the needle 54. These stops 62, 64 are used to control the amount of depth that the needle and/or fastener may be inserted into the tissue 18.

In FIG. 5A, the deployment instrument is shown ready to insert a fastener 10 into layers of tissue 18 with the tip of the instrument 50 placed against the tissue. First, the stop 62 is engaged against the head portion 55 of the needle 54, such that the needle 54 and plunger 52 can be inserted into the tissue 18 in unison. The needle 54 and plunger 52 are inserted until the head portion 55 of the needle 54 rests upon the head portion 57 of the sleeve 51, as shown in FIG. 5B. It should be apparent that if the needle is inserted into a blood vessel, as shown in FIGS. 5A-5D, care should be taken not to insert the needle past the opposite wall 17 of the vessel.

In FIG. 5C, the stop 62 is swung to engage the stop 56 on the sleeve 51. This will enable the needle 54 to be raised while the plunger 52 remains still. While the needle 54 is withdrawn, the restraining force of the needle 54 upon the fastener 10 is removed and the fastener begins to form in its unstressed and undeformed shape.

In FIG. 5D, the needle 54 is raised until its head portion 55 engages stop 64. When the needle head portion 55 engages stop 64, a doctor can be certain that the needle has exited the layers of tissue 18. The lower portion of fastener 10 will now have formed itself in the shape of a coil.

In FIG. 5E, the stop 64 is swung away from the head portion 55 such that the needle 54 can be withdrawn fully. As shown, the fastener 10 begins to form in its unstressed shape as the needle 54 is removed.

FIG. 5F shows the full withdrawal of the deployment instrument 50. The fastener 10 can now fully assume its unstressed shape. It should be noted that the unstressed coils of the fastener 10 shown in FIGS. 5D through 5F are shown having an exaggerated shape for the sake of clarity. The fastener 10 more accurately would appear as shown in FIG. 1C with the coils exerting a compressive pressure upon the layers of tissue 18.

FIGS. 6A through 6F show a second embodiment of the delivery instrument 100 which can deliver any of the fasteners described herein. The plunger 102 has a head portion 110 having both a short stop 114 and a long stop 112 attached to it. The head portion 105 of the needle 104 has two slots 116 and 118 to accept the long 112 and short 114 stops, respectively, at different times of the process. The needle 104 is slidingly accepted by sleeve 101 having a head portion 107. The tip of the delivery instrument 100, fastener 10 and needle 104 for FIGS. 6A-6F appear the same as in FIGS. 5A-5F, respectively, and are not shown for the sake of clarity.

First, as shown in FIG. 6A, the long stop 112 is brought into contact with the head portion 105 of the needle 104. The plunger 102 and needle 104 are then inserted into the tissue in unison by pushing down in the direction of arrow 120 until the needle's head portion 105 comes into contact with the sleeve's head portion 107, as shown in FIG. 6B. The needle 104 and fastener have penetrated the layers of tissue.

The head portion 110 of the plunger 102 is then rotated as shown in FIG. 6C in the direction of arrow 122 until the long stop 112 can be inserted into slot 116. The needle's head portion 105 is then raised in the direction of arrow 124 (FIG. 6D) until the needle's head portion 105 comes into contact with the short stop 114, as shown in FIG. 6D. In FIG. 6D, the needle 104 will be fully withdrawn from the layers of tissue.

In FIG. 6E, the plunger's head portion 110 is rotated in the direction of arrow 126 until the short stop 114 can be inserted into slot 118. The needle's head portion 105 is then fully raised in the direction of arrow 128 (FIG. 6F) until the head portion 105 comes into contact with the plunger's head portion 110. The needle 104 is now fully retracted from the fastener which should be fastened in the tissue and formed in its unstressed state.

It should be apparent that many types of stops could be used to position the needle 54, 104 and plunger 52, 102 of the deployment instruments 50, 100, 105. For example, the needle could function with only a single stop attached to the shaft of the plunger. Alternatively, visual indicators could be used, but would be inherently less reliable. It should be apparent that the delivery instruments as shown in FIGS. 5A-5F and 6A-6F could function properly without the short stops 64, 114, but not as reliably. Also, the delivery instruments, as shown in FIGS. 5A-5F and 6A-6F, could function without the sleeve 51 or 101, respectively. It should be apparent that a plurality of any of these deployment instruments described herein could be integrated in a single deployment instrument for sequential or simultaneous deployment of the fastener.

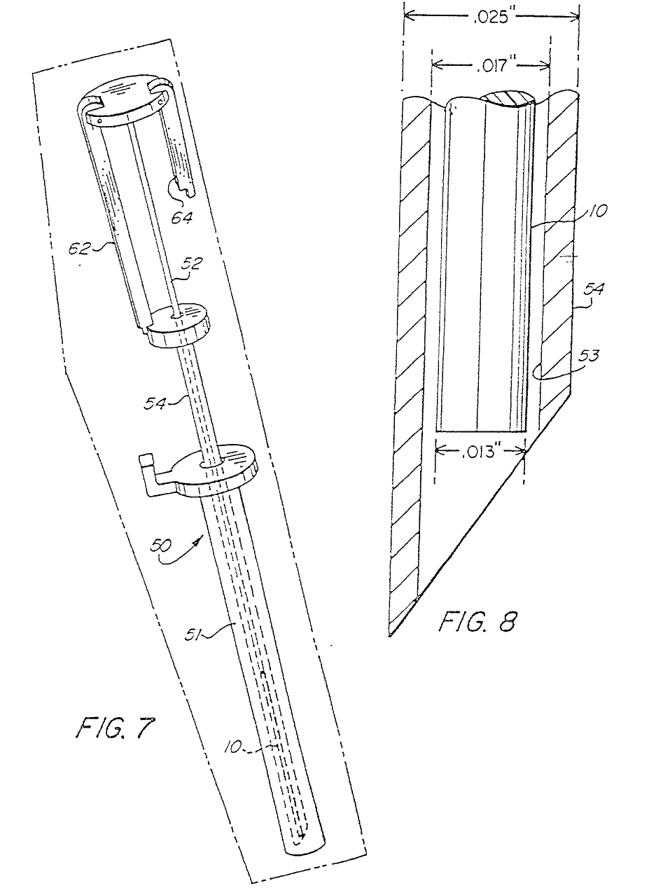

FIG. 7 shows the deployment instrument 50 as it might be shipped from a manufacturer. The surgical fastener 10 preferably is already inserted and straightened inside of the needle 54 for ease of use. The deployment instrument 50 can be shipped with or without the sleeve 51, which can be added later when the fastener is ready to be inserted.

FIG. 8 shows an enlarged view of the needle of either FIGS. 5A-5F or 6A-6F with a fastener inside of it. A typical aspect ratio of the length to diameter for this device can be in the order of 40 or 50 for less invasive use. The diameter of the fastener is preferably between 0.012 to 0.014 of an inch, more preferably its diameter is 0.013 of an inch, the inside diameter of the lumen 53 of the needle 54 is preferably 0.017 of an inch and the outside diameter of the needle is preferably 0.025 of an inch.

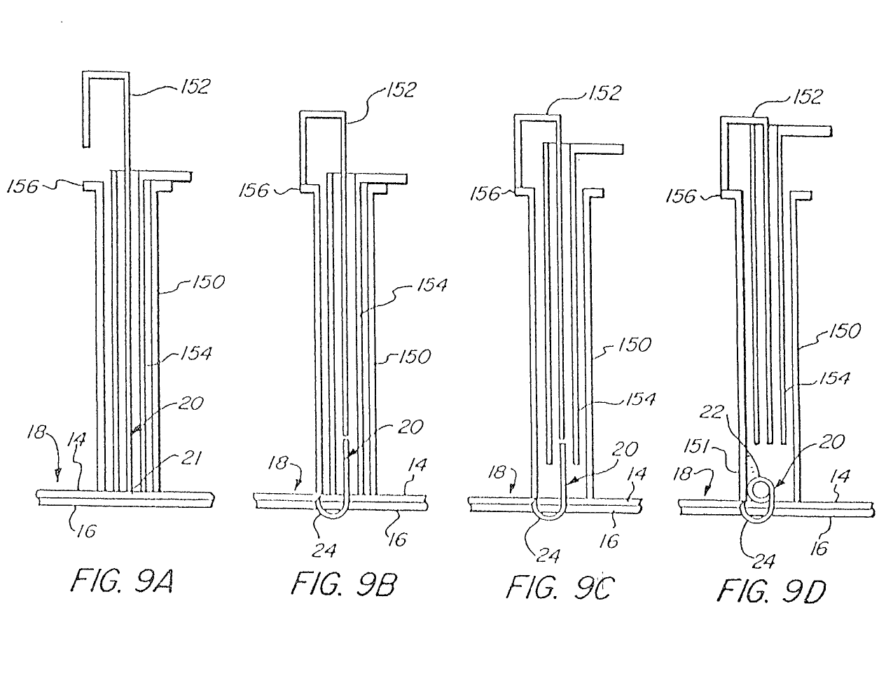

FIGS. 9A-9D show a third embodiment of the deployment instrument 150 and the method for inserting the fastener. The third embodiment of the deployment instrument 150 is different from the first two embodiments in that a restraining tube 154 is not sharpened to penetrate tissue. Thus, the surgical fastener 20 used with the deployment instrument 150 should have a sharpened end to penetrate tissue. The deployment instrument 150, consisting of slender tubes and rods, is inherently small in diameter compared to its length. Thus, FIGS. 9A-9D are illustrated with a much less favorable aspect ratio for the sake of clarity. A typical aspect ratio of the length to diameter for this device can be in the order of 40 or 50 for less invasive use. It should be apparent that other ergonomically sophisticated designs for the deployment instrument 150 can be envisioned and realized. It should also be apparent that several of these deployment instruments could be integrated in a single deployment instrument 150 for sequential or simultaneous deployment of the fastener.

FIG. 9A shows a deployment instrument 150 resting on layers of tissue 18 to be joined. The deployment instrument 150 restrains a fastener by placing stress upon it. The fastener 20, which in this example is the fastener of FIG. 1, resides in a substantially straightened form entirely within the restraining tube 154. It should be apparent that any of the fasteners described herein if given a pointed end 21 can be used with the deployment instrument of FIGS. 9A-9D. The pointed end 21 of the fastener 20 is facing toward the tissue. A plunger 152 rests on the fastener 20 and is configured to push the fastener partially out of the restraining tube 154 until the plunger 152 stops against a shield 156 as shown in FIG. 9B.

FIG. 9B shows the fastener 20 partially installed by the plunger 152. As the fastener emerges from its restraining tube, the fastener 20 penetrates the proximal 14 and distal 16 layers of tissue and gradually assumes the remembered shape of its lower coil, piercing the distal tissue layer 16 again as it turns upward. The lower coil 24 of the fastener 20, however, preferably remains substantially on the distal side of the tissue. At this point, plunger 152 bears on the shield 156 and can progress no further. Depending on the clinical application, it may be necessary to support the tissue 18 distally during penetration.

FIG. 9C shows restraining tube 154 moving upward, gradually freeing the fastener 20 to assume its remembered shape. It will obviously not be able to do so until the restraining tube 154 is completely clear, which happens when the restraining tube stops against plunger 152. The restraining tube 154 tends to pull the fastener 20 out of the tissue due to friction producing forces exerted by the fastener on the restraining tube as the former tries to assume its remembered shape. This tendency is offset by the plunger 152 bearing on the upper end of the fastener as the restraining tube 154 moves upward.

FIG. 9D shows restraining tube 154 in its fully upward position as determined by the plunger 152. The restraining tube 154 has cleared the fastener 20 and allowed it to assume its remembered, coiled shape 22, bearing against the tissue 18. The fastener 20 forms within a guide tube 151, suggesting that the guide tube 151, properly shaped, may serve to guide the fastener 20 as it forms above the tissue 18. This may be a useful feature, especially for more complex fasteners which may re-form incorrectly when released from constraint.

The guide tube 151 can serve a dual function as described above, providing a reference stop for plunger 152 and a forming guide for the fastener 20. In some cases the guide tube 151 will not be required.

The present invention also provides a system for improving fixation of endovascular grafts used to treat aortic aneurysms or occlusive disease of the aorta. In addition, present invention may be used to treat acute and chronic dissections of the aorta including those of the arch, thoracic and abdominal aorta.

More particularly, medicinal therapy for aortic aneurysms is totally ineffective. In the last 40 years, the incidence of aortic aneurysms has increased by as much as 300%, despite better control of hypertension and the risk factors of atherosclerosis.

Standard surgical repair of aortic aneurysms is by open repair. This requires a large incision for access, with a morbidity rate as high as 15-30% of patients and a mortality for elective repair of abdominal aortic aneurysms from 2-5%. With intensive care requirements and a long hospital stay of 7-14 days, surgical repair of abdominal aortic aneurysms can result in hospital charges of up to $40,000. In addition, the total recovery time is approximately 4-6 weeks.

Endovascular grafting was developed to provide a minimally invasive alternative to surgery. There are two FDA approved devices currently available for patient implantation in the United States. Many companies are developing new endovascular grafts and many of these are in clinical trials. Only one device uses hooks which imbed in the aortic wall to fix the proximal end of the graft to the aorta. The other device is reliant on stent technology, which provides fixation of the graft to the aorta by friction.

Fixation of the graft to the neck of the aneurysm is critical. Failure to achieve fixation prevents complete exclusion of the blood flow from the aneurysm sac. Thus the sac remains pressurized,with normal systematic blood pressure, which will result in enlargement and eventual rupture of the aneurysm. Because fixation of the graft is frequently dependent on friction, the length of normal aorta below the renal arteries (the neck) is the limiting factor in the successful deployment of these new graft devices. In general, the neck needs to be approximately 14-20 mm in length for successful deployment. Other factors limiting adequate apposition using stent technology include the size of the neck, whether it has a regular circumference or whether it bulges, and the angle between neck and the aneurysm.

With the above restrictions, and despite multiple technological innovations, only approximately 30-40% of patients with infrarenal abdominal aortic aneurysms are suitable candidates for endovascular techniques. The present invention provides an alternative method of fixation which is similar to the interrupted suture used by surgeons at open surgery and will significantly increase the potential patient pool able to undergo repair of the aneurysm by these minimally invasive devices.

Aortic dissection and dissecting aortic aneurysms are the most serious forms of aortic disease. In its acute stage, death may occur suddenly or within the first few hours or days after onset. Aortic dissection is characterized by a longitudinal separation within the layers of the aortic wall that extends parallel to its lumen. This separation usually arises from a tear that involves approximately 50% of the inner aortic circumference. The tear which marks the beginning of the dissection is located in the ascending arch in 68%, the transverse arch in 10%, the descending thoracic aorta in 20%, and the abdominal aorta in 2%, of patients. Surgical treatment is more difficult than other diseases of the aorta. The pathologic processes involved are more complex, more diffuse and frequently do not permit complete eradication of the disease. The aortic tissues in the acute stage are diffusely inflamed, more friable and less susceptible to secure suture. Total replacement, necessary to eradicate the process in the acute stage, is impractical and unsafe. Associated irreversible complications increase the risk and limit the incidence of a successful operation. Operation in the acute stage is limited to the aortic segment from which immediate complications arise and may be palliative rather than curative. More extensive curative replacement of the entire aorta is feasible in the more chronic stage but is still limited by the associated co-morbidity of the patient. New interventions using endovascular graft stenting have proven feasible and appear to reduce the patient morbitity in carefully selected cases. With the present invention, direct treatment of the tear and the dissection are possible.

The present invention provides a system for improving fixation of endovascular grafts used to treat aortic aneurysms or occlusive disease of the aorta. In addition, the present invention may be used to treat acute and chronic dissections of the aorta including those of the arch, thoracic and abdominal aorta. Unlike the most commonly used stent technology, which attaches the ends of the graft to the aorta or iliac vessels by friction, this system allows delivery of a special surgical fastener which penetrates through the graft and aorta to securely attach the graft to the aorta. By ensuring a direct and secure graft-aorta attachment, it is possible to sidestep the traditional requirement of a minimum length of normal artery (“neck of the aneurysm”) adjacent to the attachment site. This eliminates the anatomical limitations for endovascular grafting by the friction (stent) method. In those cases of aortic dissection, the delivery of the special surgical fastener through all layers of the aortic wall allows the re-approximation and adherence of these dissected and disrupted layers in a simple, safe and secure fashion without the need for graft placement either by open or endovascular means.

The present invention comprises a delivery catheter which is able to deploy the special fasteners from within the blood vessels to penetrate through the wall of the blood vessel, allowing attachment of an endovascular graft, including both a virgin endovascular graft and an endovascular graft previously deployed, as well as in the treatment of acute and chronic aortic dissection.

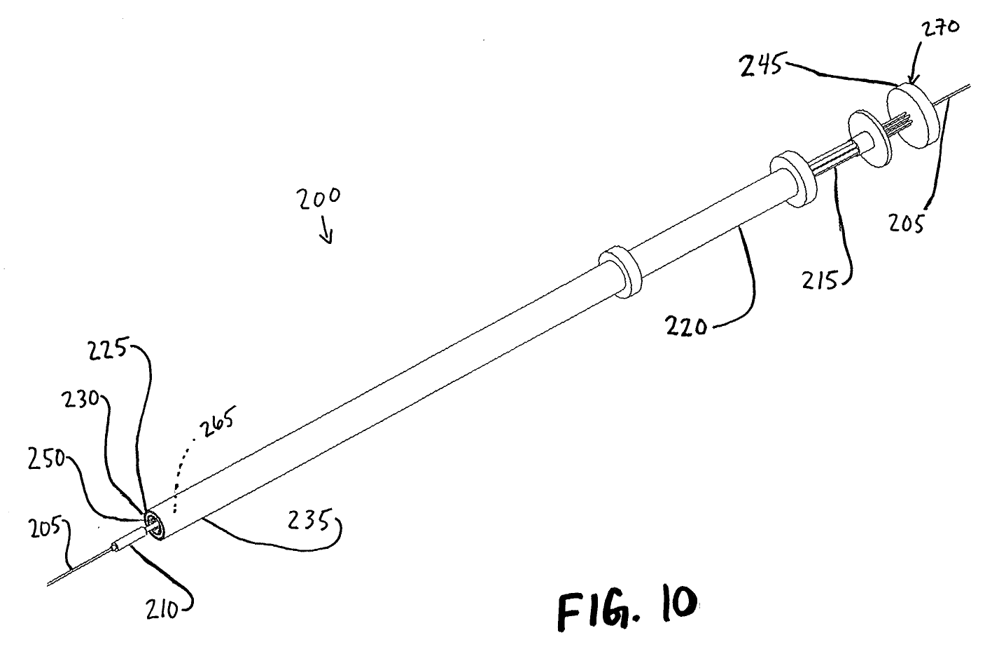

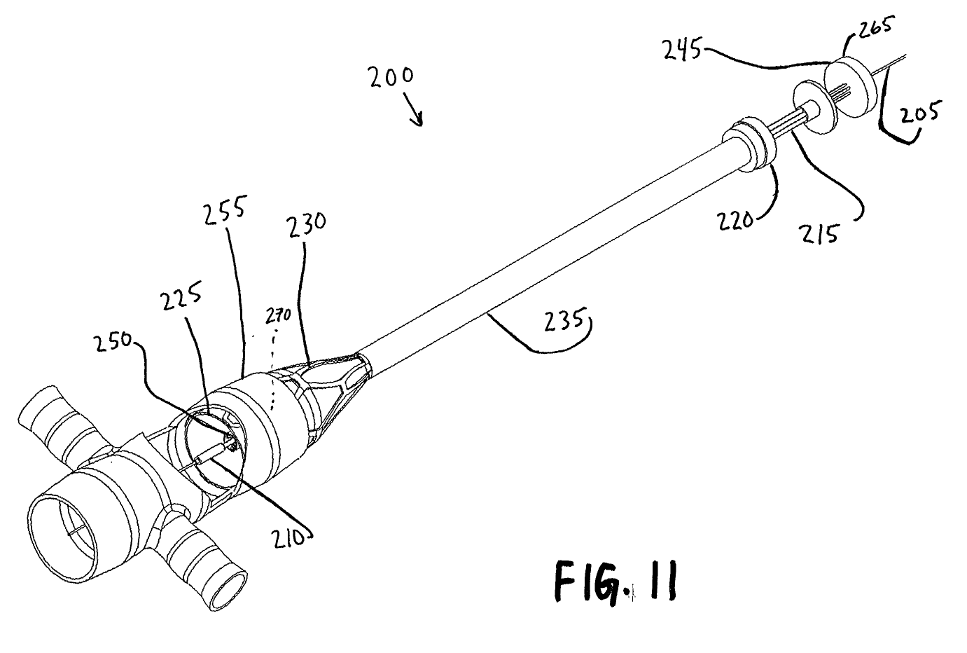

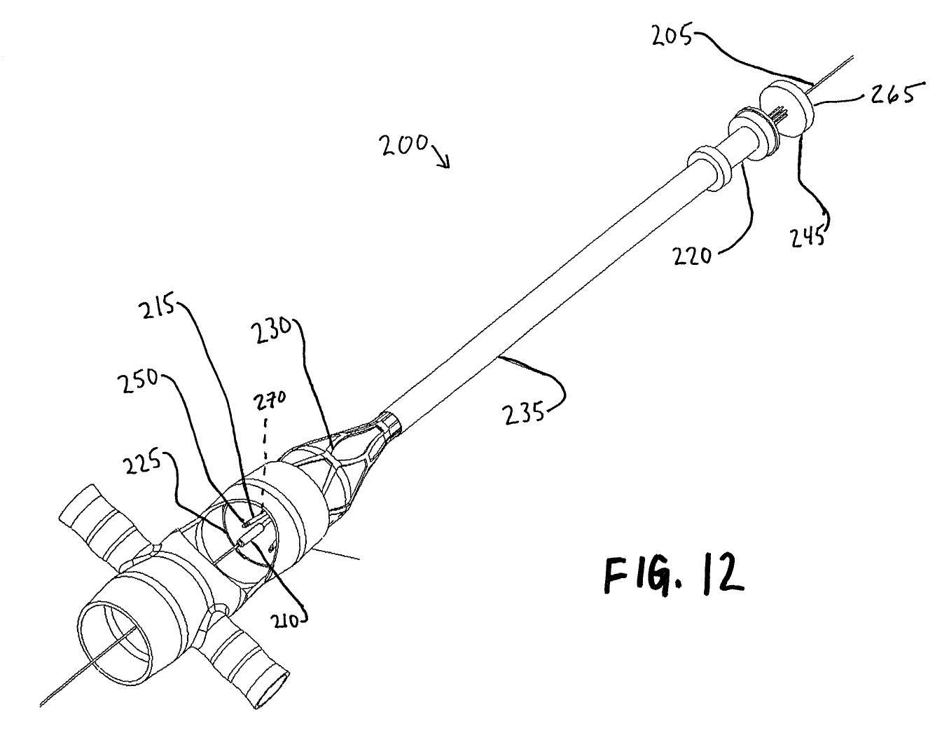

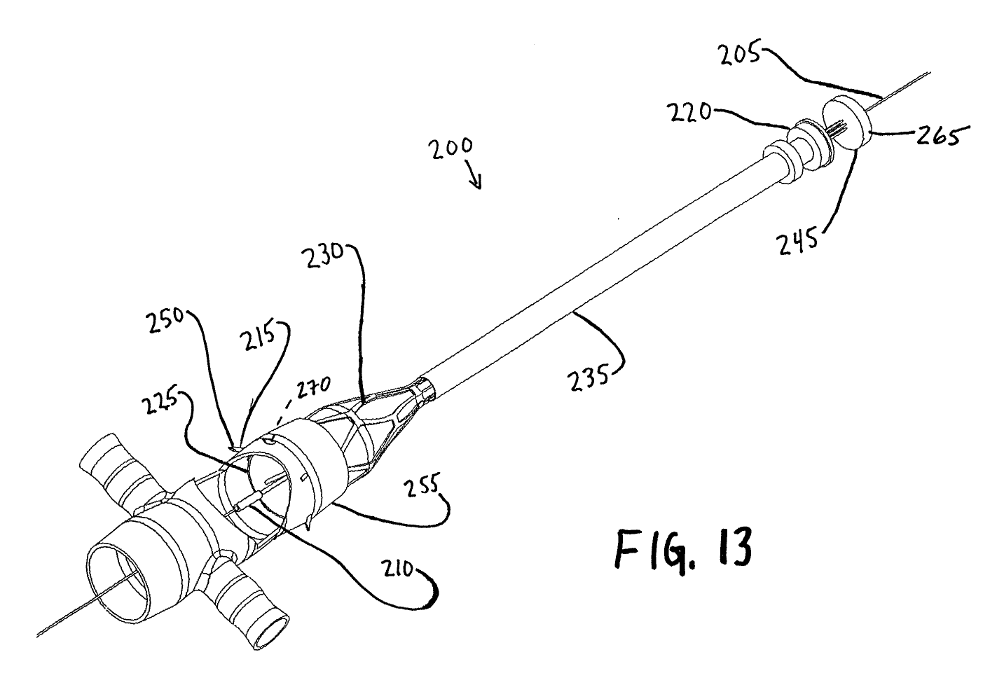

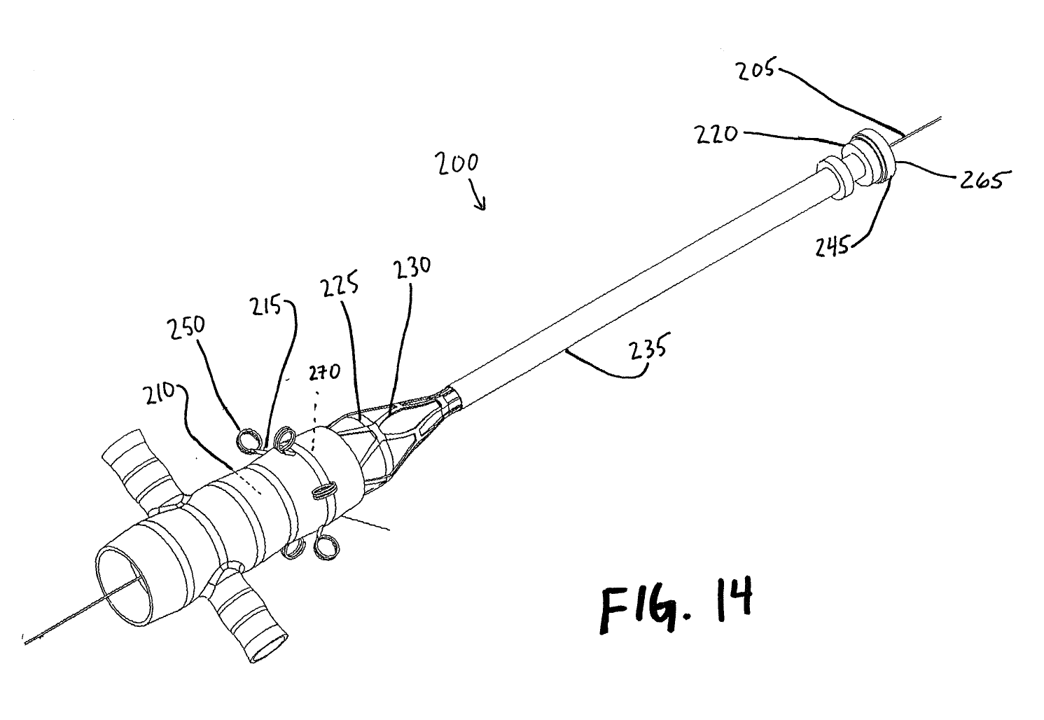

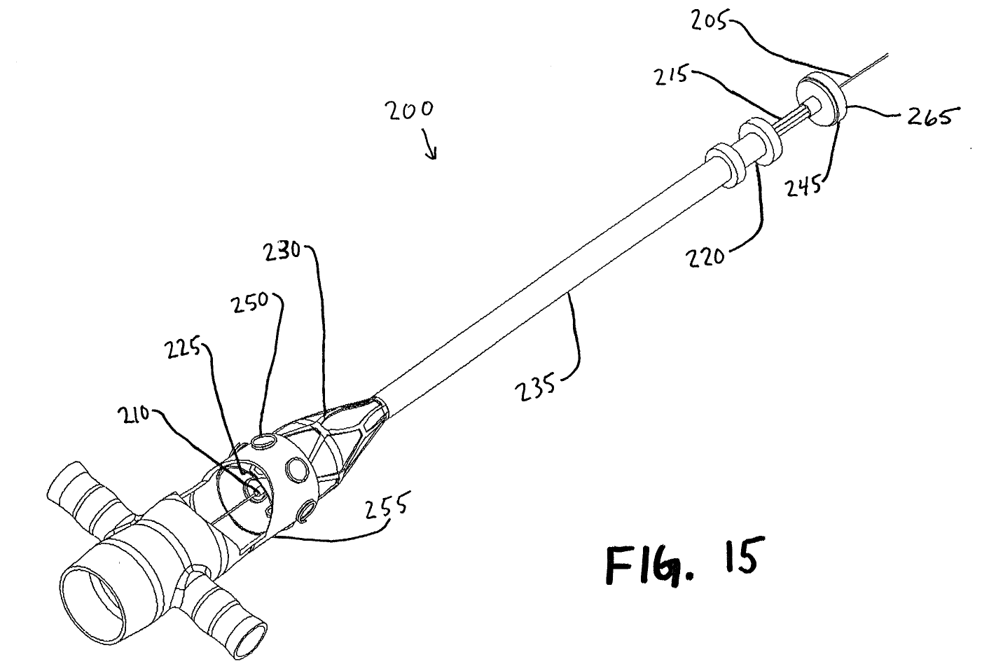

Looking now at FIGS. 10-21, another preferred embodiment of the invention is shown including an endovascular grafting and repair system 200 and a method for delivery of fasteners using system 200. In this preferred embodiment of the present invention, endovascular grafting and repair instrument 200 includes a guide wire 205, a balloon catheter 210, delivery tubes 215 (FIG. 12), a delivery tube deployment means 220 (shown as an inner sheath 220), an endovascular graft 225, a stent 230, an outer endovascular graft delivery sheath 235, a plunger 245, and fasteners 250 (FIG. 14). System 200 may be used to secure graft devices to the interior of a vascular structure, such as graft devices that rely on friction or hook technology to fix the proximal end of an endovascular graft to the interior of a vascular structure.

Still looking at FIGS. 10-21, guide wire 205 is shown supporting balloon catheter 210 to allow placement of endovascular grafting and repair system 200 in a vessel 255 (FIG. 11). Generally, guide wire 205 is a stiff wire. In the preferred embodiment of the invention, vessel 255 is shown and discussed in the context of an aorta 255, but is not limited to such a vessel. Balloon catheter 210 may provide intra-operative angiography to monitor deployment of fasteners 250 and balloon infiltration to ensure full expansion of endovascular graft 225 after attachment to the wall of aorta 255. Such balloon infiltration also provides excellent apposition of graft 225 to aorta 255.

Still looking at FIGS. 10-21, delivery tubes 215 are shown in surrounding configuration to guide wire 205. Delivery tubes 215 are preferably composed of a super-elastic material, such as Nitinol, and are restrained by inner sheath 220 in a stressed and deformed shape. This deformed shape is of a substantially linear configuration and parallel to guide wire 205 (see FIGS. 11, 16, 17 and 18). In a preferred embodiment of the invention, six to eight delivery tubes 215 are provided, and each one contains a preformed fastener 250, as described herein. Delivery tubes 215 are preformed to return to a given angle relative to guide wire 205 after being deployed from inner sheath 220 (see FIGS. 12, 13 and 19-21). This angle is to some extent dependent on the diameter of the neck of aorta 255 proximal to an aneurysm (not shown) being repaired.

Referring now to FIGS. 12, 13, 14 and 16-21, ends 260 of delivery tubes 215 are shown sharpened with a cutting edge for easier penetration through graft 225 and aorta 255. In an alternative preferred embodiment of the present invention, fasteners 250 have a sharpened end (not shown) to penetrate graft 225 and aorta 255. Delivery tubes 215 are advanced as a unit to penetrate graft 225 and aorta 255 at a predetermined distance once the site of fixation is determined and, if applicable, graft 225 is deployed. Inner sheath 220 confines delivery tubes 215 until deployment (see FIGS. 11, 16 and 18). The position of inner sheath 220 relative to the ends 260 of delivery tubes 215 helps control the angle assumed by the deployed portion of the delivery tubes 215. This positioning is accomplished by withdrawing and advancing inner sheath 220 away from and toward ends 260.

Still looking at FIGS. 10-21, stent 230 is shown surrounding at least a portion of graft 225, and outer endovascular graft delivery sheath 235 is shown as a slideable cover over the underlying system. Endovascular graft 225 may be made from various materials which include, but are not limited to, Dacron/PTFE. Graft 225 may also be surrounded by an attached stent “exoskeleton” such as is shown in the preferred embodiment. Stent 230 is part of graft 225 and may be a complete or partial stent “exoskeleton”. Outer endovascular graft delivery sheath 235 covers the underlying system for passage through blood vessels and accurate placement of the system.

Referring now to FIGS. 10-15, plunger 245 is shown having a proximal end 265 and a distal end (not shown, located adjacent to a fastener 250 located at the distal end 260 of a delivery tube 215). Plunger 245 is configured for delivery of fasteners 250 once delivery tubes 215 have penetrated aorta 255. The portion of fastener 215 placed on the distal side of aorta 255 is delivered by moving distal portion 265 of plunger 245 a predetermined distance toward ends 260 of delivery tubes 215. The portion of fastener 250 placed on the proximal side of aorta 255 is subsequently deployed by withdrawing delivery tubes 215 away from aorta 255 and away from fastener 250 in the wall of aorta 255. In addition, the withdrawal of delivery tubes 215 away from the wall of aorta 255 further decreases the length of delivery tube 215 surrounding fastener 250 while plunger 245 remains at a fixed location relative to the wall of aorta 255.

Endovascular grafting and repair system 200 is preferably used in the following manner to deliver a graft (i.e., endovascular graft 225 and stent 230) to the interior of a vascular structure (e.g., aorta 255). First, guide wire 205 is positioned in the aorta. Then the remainder of the system, encased in outer sheath 235, is moved down guide wire 205 until graft 225 is properly positioned in the aorta. Then outer sheath 235 is withdrawn, allowing graft 225 and stent 230 to deploy against the interior of aorta 255. Then inner sheath 220 is withdrawn, allowing delivery tubes 215 to angulate outward. Next, inner sheath 220 and delivery tubes 215 are advanced distally, causing the sharp distal ends 260 of delivery tubes 215 to penetrate through graft 255, stent 230 and the walls of aorta 255. As this occurs, delivery tubes 215 carry fasteners 250 outward so that portions of fasteners 250 also extend through graft 225, stent 230 and aorta 255. Then plunger 245 is advanced so as to deploy the outer ends of fasteners 250 against the outside wall of aorta 255. Next, delivery tubes 215 are retracted, thereby causing the inner ends of fasteners 250 to be deployed against the inside of graft 225. As a result, graft 225 and stent 230 will be secured to aorta 255 by the coils 12 of fasteners 250. Then balloon catheter 210 is inflated so as to ensure full expansion of graft 225 and stent 230, whereby to ensure close apposition of the graft to the aortic wall.

It should also be appreciated that system 200 can be used to secure a previously-deployed endovascular graft to the wall of an aorta. More specifically, in some situations a previously-deployed endovascular graft may be in danger of migrating within the aorta. In this case system 200 (without graft 225, stent 230 and inner sheath 220) may be used to set fasteners 250 through the previously-deployed graft, whereby to ensure proper fixation of the graft relative to the aorta.

It should be understood that the foregoing is illustrative and not limiting and that modifications may be made by those skilled in the art without departing from the scope of the invention.

What is claimed is:

1. Apparatus for inserting a surgical fastener through a plurality of portions of material from within an endovascular pathway, said apparatus comprising:
a surgical fastener having first and second ends and made from a material which enables said fastener to be transformed from a first stressed elongate shape to a second unstressed shape upon the release of said fastener from a stressed condition, said first stressed elongate shape of said fastener enabling said first end to be extended through a plurality of layers of material, and with said second shape of the fastener being in the form of a spring with a plurality of coils around a spring axis, with said coils being spring biased towards each other along said spring axis with sufficient axial force so as to enable coils on opposite sides of layers to clamp the layers of material together along the spring axis;
a delivery tube having third and fourth ends, first and second tube portions adjacent to said third and fourth ends, respectively, and forming a longitudinal axis between the third and fourth ends, said delivery tube including a material which enables transformation from a third stressed elongate shape to a fourth unstressed shape upon the release from a stressed condition to an unstressed condition, said third stressed elongate shape enabling said third end to be extended through an endovascular pathway, said fourth unstressed shape being formed with said first and second tube portions being configured at an angle to one another;
delivery tube deployment means being configurable between a first position and a second position, said first position of said delivery tube deployment means restraining said delivery tube in said third stressed elongate shape, and said second position of said delivery tube deployment means releasing said delivery tube in the fourth unstressed shape;
penetration means adjacent said third end of said delivery tube, said penetration means being configured to pierce through a vascular structure in the endovascular pathway; and
insertion means adjacent to said first end of said delivery tube, said insertion means being configured to place said surgical fastener through the vascular structure pierced by said penetration means.

2. Apparatus for endovascular surgery according to claim 1 further comprising a plurality of delivery tubes and each of said delivery tubes being controlled by said delivery tube deployment means.

3. Apparatus for endovascular surgery according to claim 1 wherein said material has super-elastic properties.

4. Apparatus for endovascular surgery according to claim 3 wherein said super-elastic material is Nitinol.

5. Apparatus for endovascular surgery according to claim 1 wherein said penetration means is a sharpened cutting edge formed on said third end of said delivery tube.

6. Apparatus for endovascular surgery according to claim 1 wherein said penetration means is a sharpened cutting edge formed on said first end of said surgical fastener.

7. Apparatus for endovascular surgery according to claim 1 wherein the angle of said second unstressed shape of said delivery tube formed with said first and second tube portions angled to one another is dependent on the diameter of said vascular structure of the endovascular pathway.

8. Apparatus for endovascular surgery according to claim 1 wherein said insertion means is a plunger being configured within said delivery tube, said plunger having first and second portions, said first and second portions being configured adjacent said third and fourth ends of said delivery tube, respectively, said first end of said plunger being configured adjacent said second end of said fastener, whereby movement of said plunger a predetermined distance toward the third end of said delivery tube forces said fastener through said vascular structure a distance corresponding to said predetermined distance.

9. Apparatus for endovascular surgery according to claim 1 further comprising a guide wire having a given stiffness for allowing positioning within the endovascular pathway of said vascular structure, said guide wire having a longitudinal axis, said first stressed elongate shape of delivery tube being configured in parallel to said guide wire.

10. Apparatus for endovascular surgery according to claim 9 further including a balloon catheter supported by said guide wire.

11. Apparatus for endovascular surgery according to claim 10 wherein said balloon catheter provides a reference for the proper placement of said fasteners.

12. Apparatus for endovascular surgery according to claim 1 wherein said delivery tube deployment means is an inner sheath having first and second ends, being in surrounding configuration parallel to said longitudinal axis of, and along a portion of, said delivery tube, being in slideable configuration from a first distance to a second distance from said third end of said delivery tube, wherein withdrawal away from said third end and advancement toward said third end of said inner sheath controls the angle of said delivery tube.

13. Apparatus for endovascular surgery according to claim 1 further comprising an endovascular graft being in surrounding configuration to said third end of said delivery tube wherein said surgical fastener delivered by said delivery tube attaches said endovascular graft to the vascular structure in the endovascular pathway.

14. Apparatus for endovascular surgery according to claim 13 wherein said apparatus further includes a balloon catheter supported by a guide wire, and further wherein said balloon catheter provides balloon inflation to ensure full expansion of said graft to the wall of said vascular structure.

15. Apparatus for endovascular surgery according to claim 13 wherein said endovascular graft is constructed of Dacron/PTFE material.

16. Apparatus for endovascular surgery according to claim 13 wherein said endovascular graft is at least partially surrounded by a stent.

17. Apparatus for endovascular surgery according to claim 16 wherein said stent is a partial exoskeleton surrounding said endovascular graft.

18. Apparatus for endovascular surgery according to claim 16 wherein said stent is a complete exoskeleton.

19. Apparatus for endovascular surgery according to claim 1 further comprising an outer endovascular delivery sheath being in slideable, surrounding configuration to selectively cover a portion of said delivery tube from said third end to said fourth end.

20. Apparatus for endovascular surgery according to claim 1 wherein the vascular structure is an aorta.

21. A method for inserting a surgical fastener through a plurality of portions of material from within an endovascular pathway, said method comprising:
providing apparatus for inserting a surgical fastener through a plurality of portions of material from within an endovascular pathway, said apparatus comprising:
a surgical fastener having first and second ends and made from a material which enables said fastener to be transformed from a first stressed elongate shape to a second unstressed shape upon the release of said fastener from a stressed condition, said first stressed elongate shape of said fastener enabling said first end to be extended through a plurality of layers of material, and with said second shape of the element being in the form of a spring with a plurality of coils around a spring axis, with said coils being spring biased towards each other along said spring axis with sufficient axial force so as to enable coils on opposite sides of layers to clamp the layers of material together along spring axis;
a delivery tube having third and fourth ends, first and second tube portions adjacent to said third and fourth ends, respectively, and forming a longitudinal axis between the third and fourth ends, said delivery tube including a material which enables transformation from a third stressed elongate shape to a fourth unstressed shape upon the release from a stressed condition to an unstressed condition, said third stressed elongate shape enabling said third end to be extended through an endovascular pathway, with said fourth unstressed shape being formed with said first and second tube portions being configured at an angle to one another;
delivery tube deployment means being configurable between a first position and a second position, said first position of said delivery tube deployment means restraining said delivery tube in said third stressed elongate shape, and said second position of said delivery tube deployment means releasing said delivery tube in said fourth unstressed shape;
penetration means adjacent said third end of said delivery tube, said penetration means being configured to pierce through a vascular structure in the endovascular pathway; and
insertion means adjacent to said first end of said delivery tube, said insertion means being configured to place said surgical fastener through the vascular structure pierced by said penetration means;
placing said delivery tube adjacent said vascular structure, with said delivery tube being configured in said third stressed elongate shape;
deploying said delivery tube from said third elongate shape to said forth elongate shape with said delivery tube deployment means, said deployment of said delivery tube placing said third end adjacent to the vascular structure in the endovascular pathway;
penetrating the vascular structure in the endovascular pathway with said penetration means, said penetration of the vascular structure being performed at said third end of said delivery tube; and
inserting said surgical fastener through the plurality of portions of material using said insertion means, said insertion of said surgical fastener being performed from inside of said vascular structure.

22. A method according to claim 21 wherein the step of placing said delivery tube adjacent said vascular structure includes using a guide wire to position said delivery tube.

23. A method according to claim 21 wherein the said delivery tube deployment means is an inner sheath having first and second ends, being in surrounding configuration parallel to said longitudinal axis of, and along a portion of, said delivery tube, being in slideable configuration from a first distance to a second distance from said third end of said delivery tube, wherein withdrawal away from said third end and advancement toward said third end of said inner sheath controls the angle of said delivery tube, and the steps of deploying said delivery tube from said third elongate shape to said fourth elongate shape includes withdrawal of said inner sheath away from said third end of said delivery tube, and advancement of said inner sheath toward said third end of said delivery tube returns said delivery tube from said fourth elongate shape to said third elongate shape.

24. A method for according to claim 23 wherein the step of deploying said delivery tube from said third elongate shape to said fourth elongate shape is an incremental process and is directly proportional to the distance said inner sheath is withdrawn relative to said third end of said delivery tube.

25. A method according to claim 21 wherein said penetration means used in the step of penetrating the vascular structure in the endovascular pathway is a sharpened cutting edge formed on said third end of said delivery tube.

26. A method according to claim 21 wherein said penetration means used in the step of penetrating the vascular structure in the endovascular pathway is a sharpened cutting edge formed on said first end of said surgical fastener.

27. A method according to claim 21 wherein said insertion means used in the step of inserting said surgical fastener through the plurality of portions of material is a plunger sized to slidingly move through said delivery means to advance said surgical fastener toward said third end of said delivery tube.

28. A method according to claim 21 further comprising the step of withdrawing said delivery tube away from the plurality of portions of material to release said surgical fastener from said stressed condition on said second end of said surgical fastener whereby said surgical fastener clamps the plurality of layers of the material together.

29. A method according to claim 21 wherein one of said plurality of portions of material comprises a vascular structure, and further wherein another of said plurality of portions of material comprises a graft.

30. A method according to claim 29 wherein said apparatus for inserting a surgical fastener is positioned in the vascular structure prior to placement of said graft adjacent to said vascular structure.

31. A method according to claim 29 wherein said graft is placed in said vascular structure prior to positioning said apparatus for inserting a surgical fastener in said vascular structure.
